(19) United States
(12) Patent Application Publication
(10) Pub. No.: US 2026/0306528 A1
(43) Pub. Date: Oct. 8, 2026

(54) HYDROGEL INK, HYDROGEL AND USES THEREOF

(71) Applicants: Nanyang Technological University, Singapore (SG); National University of Singapore, Singapore (SG)

(72) Inventors: Sing Yian CHEW, Singapore (SG); Chongquan HUANG, Singapore (SG); Kieran Yan Ki LAU, Singapore (SG); Thi Nguyet Minh Le, Singapore (SG); Migara Kavishka JAYASINGHE, Singapore (SG)

(21) Appl. No.: 19/485,076

(22) PCT Filed: May 17, 2024
(86) PCT No.: PCT/SG2024/050330

§ 371 Date: Nov. 17, 2025

(30) Foreign Application Priority Data
May 18, 2023 (SG) 10202301395R

(51) Int. Cl. A61K 31/711; A61K 9/06; A61K 9/48; A61K 31/7105; A61K 47/08; A61K 47/42; A61P 25/00; C12N 15/63

(52) U.S. Cl. CPC A61K 31/711; A61K 9/06; A61K 9/4858; A61K 9/4866; A61K 31/7105; A61K 47/08; A61K 47/42; A61P 25/00; C12N 15/63

(57) ABSTRACT

This disclosure concerns a hydrogel ink comprising lipid particles, polynucleotides loaded within the lipid particles and a bioink. In one embodiment, the lipid particles comprise extracellular vesicles. The disclosure also concerns the hydrogel thereof, and its use in treating a neural disease or condition, or regenerating a soft tissue.

### TECHNICAL FIELD The present invention relates, in general terms, to hydrogel ink, hydrogel and uses thereof.

### BACKGROUND

Nucleic acid therapy, especially RNA interference, is a promising treatment method that has been widely explored for cancer therapy and genetic disease treatment. It also holds great potential for tissue regeneration. For example, RNAi can improve the cell differentiation and regeneration capacity and deplete detrimental factors in injury sites to facilitate cell/tissue survival and regeneration.

However, efficient delivery system is required for practical use in tissue regeneration, and the requirements for such delivery systems are different from systemic delivery approaches seen in cancer therapy and genetic disease treatments.

Theoretically, an ideal delivery system should be able to help gene material to pass through the cell membrane and allow downstream function, keep the gene material from degradation by endogenous enzymes, enable long-term and sustained release of RNA to cover the process of tissue regeneration, and/or be biocompatible in physical and chemical properties to allow tissue regeneration.

Spinal cord injury (SCI) results in severe morbidity and permanent disability. Globally, between 250,000 and 500,000 patients, each year suffer a spinal cord injury. In the United States, there are approximately 17,000 new cases of SCI each year, and roughly 282,000 persons are estimated to be living with SCI. There are, however, limited therapeutic solutions for SCI.

There are currently limited delivery systems that may perform these functions, alone or in combination.

It would be desirable to overcome or ameliorate at least one of the above-described problems.

### SUMMARY

The present disclosure provides a hydrogel ink, comprising:
a) lipid particles;
b) polynucleotides loaded within the lipid particles; and
c) a bioink.

In some embodiments, the lipid particles are extracellular vesicles.

In some embodiments, the lipid particles are extracellular vesicles derived from red blood cells (erythrocytes), immune cells, endothelial cells, liver cells, epithelial cells, stem cells, Schwann cell, or a combination thereof.

In some embodiments, the lipid particles are characterised by an average particle size of about 100 nm to about 400 nm.

In some embodiments, the hydrogel ink is characterised by lipid particles loading of about 1×10⁸ particles/μL to about 1×10¹¹ particles/μL.

In some embodiments, the polynucleotides or nucleic acids are selected from deoxyribonucleic acid (DNA), small non-coding RNA, or small interfering RNA (siRNA). In some embodiments, the polynucleotides are about 0.5% wt/wt to about 10% wt/wt relative to the lipid particles.

In some embodiments, the bioink is selected from gelatin methacryloyl (GelMA), gelatin, collagen, silk fibroin, alginate, nanofibrillated cellulose, hyaluronic acid, poly(caprolactone-co-ethyl ethylene phosphate), polyethylene glycol diacrylate (PEGDA) or a combination thereof.

In some embodiments, the bioink is about 10% wt/wt to about 40% wt/wt relative to the hydrogel ink.

In some embodiments, the hydrogel ink further comprising a photoinitiator.

In some embodiments, the photoinitiator is about 0.1% wt/wt to about 1% wt/wt relative to the hydrogel ink.

In some embodiments, the hydrogel ink further comprising a photoquencher.

In some embodiments, the photoquencher is about 1×10⁻⁴% wt/wt to about 1×10⁻²% wt/wt relative to the hydrogel ink.

In some embodiments, the hydrogel ink further comprises a drug.

In some embodiments, the hydrogel ink is 3D printable.

The present disclosure also provides a hydrogel, comprising:
a) lipid particles;
b) polynucleotides loaded within the lipid particles; and
c) a bioink matrix;
   wherein the lipid particles are homogenously dispersed within the bioink matrix.

In some embodiments, the hydrogel is formed as a multichannel scaffold.

The present disclosure also provides a method of transfecting cells, comprising the step of contacting the hydrogel as disclosed herein with the cells.

In some embodiments, the method is characterised by a EV loading to 1×10⁵ cells of about 10 μg to about 100 μg.

In some embodiments, the method is characterised by a polynucleotide loading to 1×10⁵ cells of about 150 ng to about 400 ng.

In some embodiments, the method is characterised by a transfection rate of at least about 50%.

The present disclosure also provides a method of treating a neural condition or disease in a subject in need thereof, comprising administering a therapeutically effective amount of the hydrogel as disclosed herein to a subject.

The present disclosure also provides a hydrogel as disclosed herein for use in treating a neural condition or disease.

The present disclosure also provides a use of the hydrogel as disclosed herein in the manufacture of a medicament for the treatment a neural condition or disease.

In some embodiments, the neural condition or disease is selected from spinal cord injury, brain injury, ischemic stroke, and sclerosis.

The present disclosure also provides a method of regenerating a soft tissue in a subject in need thereof, comprising administering a therapeutically effective amount of the hydrogel as disclosed herein to a subject.

The present disclosure also provides a hydrogel as disclosed herein for use in regenerating a soft tissue.

The present disclosure also provides a use of the hydrogel as disclosed herein in the manufacture of a medicament for the regeneration of a soft tissue.

In some embodiments, the soft tissue is a nerve, cardiovascular tissue or skin.

### BRIEF DESCRIPTION OF THE DRAWINGS

Embodiments of the present invention will now be described, by way of non-limiting example, with reference to the drawings in which:

FIG. 1 shows EVs and EVs/GelMA scaffolds effect on neural cells. A. flowcytometry assay after adding fluorescence loaded EVs into astrocytes culture and OPCs culture. B. SiGAPDH-EVs knockdown effect on rat cortical neurons. C. siGAPDH-EVs knockdown effect on rat brain-dissociated glial cells. D. Graph abstract of scaffold manufacturing. E. Knockdown effect of siGAPDH-EVs/GelMA scaffold on OPCs. F. miR219/miR338-EVs/GelMA scaffold effect on OPCs differentiation. Left panel: Olig2 average intensity. Right panel: olig2 positive particles ratio at total DAPI particles.

FIG. 2 shows localized delivery of EVs up to 3 mm from the scaffold-tissue interface after 7 days in vivo.

FIG. 3 shows the number of CC1-positive cells over Olig2-positive cells after treatment with EV containing hydrogel in vivo.

### DETAILED DESCRIPTION

The present disclosure is predicated on the understanding that the advantages of scaffold and EVs maybe combined into an RNA delivery platform. The inventors had surveyed the landscape and found that there are currently no 3D-printed scaffolds that are capable of 1) achieving sustained delivery of RBCEV-small noncoding RNAs, 2) transfect primary neural cells 3) provide physical support and signals to direct tissue regeneration and 4) sustained gene manipulation to drive cell fate.

Current 3D cultures are limited due to potential EV-hydrogel interactions, thus there is difficulty in releasing EVs, and thus the subsequent release of polynucleotides for therapeutic purposes. The inventors had further found that certain conditions and/or parameters may modulate the EV-hydrogel interactions, thus allowing the hydrogel ink to be used in neuro-related diseases and conditions.

Accordingly, the present disclosure provides a hydrogel ink, comprising:
a) lipid particles;
b) polynucleotides loaded within the lipid particles; and
c) a bioink.

Compared with traditional scaffolds or bare RNA delivery, the present disclosure provides RNA stability, and optionally also provides control over drug release kinetics. Importantly, using a scaffold-mediated delivery approach, scaffold design parameters may also be utilised to further control gene uptake, gene silencing and cell response (e.g. through design of physical and chemical properties of the scaffolds). When compared with the direct injection of gene vectors, this platform could enable localized gene delivery to avoid systemic side effect and reduce drug dosage requirements, and provide a tunable/customizable ideal physical microenvironment for cell and tissue regrowth.

For example, the scaffolds may be produced by 3D printing the hydrogel ink. A patient who needs a scaffold may be bioimaged by the doctor (e.g. using MRI). The 3D image may then be created into a CAD file, which is the input file format for 3D printing. Based on this, the patient's defect site may be scanned and a scaffold of the right shape and size printed to fit the defect.

As used herein, “lipid particles” refer to particles which comprises lipids, which are man-made or natural. The particles may be nanoparticles or microparticles, and includes, but not limited to, liposomes, lipid nanoparticles, solid lipid nanoparticles, nanostructured lipid carriers, nanoemulsions, lipid vesicles, or their derivatives thereof.

In some embodiments, the lipid particles are extracellular vesicles. Extracellular vesicles (EVs) are cell-derived membrane-surrounded vesicles that carry bioactive molecules and deliver them to recipient cells. Classical EVs are exosomes, microvesicles, and apoptotic bodies. EVs are found to be biocompatible, and hence not toxic to the body and are less likely to provoke an immune response. They may have a long circulation time, thus improve their effectiveness as drug delivery vehicles. They may also be stable, thus may be stable in the bloodstream, which allows for them to reach target cells. EV producer cell phenotype partly determines the array of cytosolic proteins (i.e. molecular chaperones, metabolic enzymes, ribosomal proteins, etc.), cell-surface proteins, and nucleic acids that may be packaged within EVs. This diversity suggests that pleiotropic, complementary, and/or synergistic effects for therapeutics may be achieved through selection of the type of EVs or via modifications. For example, mesenchymal stem/stromal cell (MSC) EVs may provide an anti-inflammatory and pro-angiogenic effects for SCI and other types of neurotrauma.

In some embodiments, the extracellular vesicles are derived from red blood cells (erythrocytes), immune cells, endothelial cells, liver cells, epithelial cells, stem cells, Schwann cell, or a combination thereof. In some embodiments, the extracellular vesicles are derived from a non central nervous system (CNS) cell. In some embodiments, the EVs are red blood cell extracellular vesicles (RBCEVs).

In some embodiments, RBCEVs, as the primary nucleic acid carrier, are loaded with SiRNA/miRNA and may help gene material pass through the cell membrane and allow downstream function, and keep the gene material from degradation by endogenous enzymes. Thereafter, the RNA-loaded RBCEVs were incorporated into a bioink such as gelatin methacryloyl (GelMA) solution for 3D printing, which produced the final RNA-RBCEVs/GelMA scaffold for in vitro and/or in vivo transplantation.

The inventors have found RBCEVs to be advantageous over EVs from other sources in terms of biocompatibility, scalability, safety and reproducibility. RBCEV is a cell-derived natural lipid particle. RBCEVs may assist to bypass cellular barriers as well as other hurdles to drug delivery, including cytotoxicity, RNase susceptibility, phagocytosis, endosomal accumulation, multidrug resistance, and immunogenicity. RBCEVs can be readily induced and isolated from human red blood cells (RBCs) at yields far exceeding typical yields obtained from cell lines, overcoming one of the major limitations of EV-based therapies—clinical scaling and production. They may also be purified on a large scale reproducibly. RBCEVs may be produced on a large scale without the need for cell culture, thereby reducing the cost of production and the risk of contamination. Given that EVs are derived from human RBCs also ensures that their contents are safe and do not contain any genomic materials that may have adverse effects on recipient cells. As RBCs have been routinely used for blood transfusions for decades, and it is reasonable to assume that RBCEVs derived from RBCs would also carry a similar safe, non-immunogenic, non-toxic biocompatible profile. In addition, in vivo data in animal models supporting the safe and biocompatible application of RBCEVs for therapeutic use.

Further, RBCs do not contain much organelle, which means their derived EVs are cleaner. RBCs do not have nucleus, which means they are safer in case of nucleic acid contamination from unwanted DNA or RNA. RBCs membrane surface is simpler than other cell types so that they would not induce much immune response.

In some embodiments, the lipid particles are characterised by an average particle size of about 100 nm to about 400 nm. In other embodiments, the average particle size is about 120 nm to about 400 nm, about 140 nm to about 400 nm, about 160 nm to about 400 nm, about 180 nm to about 400 nm, about 200 nm to about 400 nm, about 200 nm to about 380 nm, about 200 nm to about 360 nm, about 200 nm to about 340 nm, about 200 nm to about 320 nm, or about 200 nm to about 300 nm. In other embodiments, the average particle size is about 200 nm.

In some embodiments, the lipid particles are electroporated. In some embodiments, the lipid particles are characterised by pore size of about 1 nm to about 100 nm, about 1 nm to about 90 nm, about 1 nm to about 80 nm, about 1 nm to about 70 nm, about 1 nm to about 60 nm, about 1 nm to about 50 nm, about 1 nm to about 40 nm, about 1 nm to about 30 nm, about 1 nm to about 20 nm, or about 1 nm to about 10 nm.

In some embodiments, the lipid particles are transfected with polynucleotides. The transfection may occur with the use of transfectants and or loading reagents. This was found that to be more advantageous than using an electroporation method as electroporation would render some of the polynucleotides non-biofunctional.

The lipid particles may be surface functionalized with ligands/targeting molecules using bioconjugate chemistry, enzymatic ligation, and/or affinity modifications. For example, the lipid particle may be functionalised with a stimuli responsive linker between lipid particles and scaffold to have controlled release of the particles (e.g. MMP sensitive linkages). For example, two types of lipid particles with two types of linking chemistry respectively to the scaffolds may be used, one strong linkage and one weaker linkage, to achieve sequential release of the drugs (e.g. first to tune immune response, then to enhance neuronal regeneration and then to enhance remyelination).

In some embodiments, the lipid particles are about 0.01% wt/wt to about 10% wt/wt relative to the hydrogel ink. In other embodiments, the concentration is about 0.01% wt/wt to about 5% wt/wt, about 0.01% wt/wt to about 1% wt/wt, about 0.01% wt/wt to about 0.8% wt/wt, about 0.01% wt/wt to about 0.6% wt/wt, about 0.01% wt/wt to about 0.4% wt/wt, about 0.01% wt/wt to about 0.2% wt/wt, or about 0.01% wt/wt to about 0.1% wt/wt. In other embodiments, the concentration is about 0.2% wt/wt.

In some embodiments, the hydrogel ink is characterised by a lipid particles loading of about 1×10⁸ particles/μL to about 1×10¹¹ particles/μL. In other embodiments, the lipid particles loading is about 1×10⁸ particles/μL to about 8×10¹⁰ particles/μL, about 1×10⁸ particles/μL to about 6×10¹⁰ particles/μL, about 1×10⁸ particles/μL to about 4×10¹⁰ particles/μL, about 1×10⁸ particles/μL to about 2×10¹⁰ particles/μL, about 1×10⁸ particles/μL to about 1×10¹⁰ particles/μL, about 1×10⁸ particles/μL to about 8×10⁹ particles/μL, about 1×10⁸ particles/μL to about 6×10⁹ particles/μL, about 2×10⁸ particles/μL to about 6×10⁹ particles/μL, about 4×10⁸ particles/μL to about 6×10⁹ particles/μL, about 6×10⁸ particles/μL to about 6×10⁹ particles/μL, about 8×10⁸ particles/μL to about 6×10⁹ particles/μL, or about 1×10⁹ particles/μL to about 6×10⁹ particles/μL. In some embodiments, the hydrogel ink is characterised by a lipid particles loading of about 5×10⁹ particles/μL.

In some embodiments, the polynucleotides or nucleic acids are selected from ribonucleic acid (RNA) and deoxyribonucleic acid (DNA), or a combination thereof. The RNA may be a small non-coding RNA, or small interfering RNA (siRNA). The polynucleotides may comprise about 5 to about 100 monomeric units.

The polynucleotides are encapsulated within the lipid particles. In some embodiments, the polynucleotides loaded lipid particles is characterised by a polynucleotide loading of about 0.5% wt/wt to about 10% wt/wt relative to the lipid particles. In other embodiments, the polynucleotide loading is about 0.5% wt/wt to about 9% wt/wt, about 0.5% wt/wt to about 8% wt/wt, about 0.5% wt/wt to about 7% wt/wt, about 0.5 % wt/wt to about 6% wt/wt, about 0.5% wt/wt to about 5% wt/wt, about 0.5% wt/wt to about 4% wt/wt, about 0.5% wt/wt to about 3% wt/wt, about 0.5% wt/wt to about 2% wt/wt, about 1% wt/wt to about 10% wt/wt, or about 2% wt/wt to about 10% wt/wt. In other embodiments, the polynucleotide loading is about 2% wt/wt to about 3% wt/wt. This is about 1 μg of polynucleotides in 40 μg of lipid particles.

Depending on the nature of the polynucleotide and the size, the number of polynucleotides per lipid particle can be higher than 100. In some embodiments, the polynucleotides loaded lipid particles is characterised by a polynucleotide loading of about 1 polynucleotide/lipid particle to about 600 polynucleotide/lipid particle. In other embodiments, the polynucleotide loading is about 10 polynucleotide/lipid particle to about 600 polynucleotide/lipid particle, about 100 polynucleotide/lipid particle to about 600 polynucleotide/lipid particle, about 1 50 polynucleotide/lipid particle to about 600 polynucleotide/lipid particle, about 200 polynucleotide/lipid particle to about 600 polynucleotide/lipid particle, about 250 polynucleotide/lipid particle to about 600 polynucleotide/lipid particle, about 300 polynucleotide/lipid particle to about 600 polynucleotide/lipid particle, about 350 polynucleotide/lipid particle to about 600 polynucleotide/lipid particle, about 400 polynucleotide/lipid particle to about 600 polynucleotide/lipid particle, or about 450 polynucleotide/lipid particle to about 600 polynucleotide/lipid particle.

A bioink is any natural or synthetic polymer which is selected for at least its biocompatible components, and optionally favourable rheological properties. A bioink will typically support living cells, by for example aiding cell adhesion, cell proliferation and cell differentiation during maturation. The primary purpose of bioinks is to support the growth and proliferation of cells. As such, the formulation of the bioink should be conducive to cell survival for the duration of the culture period, by providing a suitable environment for cells to adhere, grow, and differentiate. Additionally, bioinks should maintain the 3D structure during and after printing. This involves a careful balance of mechanical properties to ensure the bioink has the necessary strength and flexibility. The bioink should also be biocompatible, causing no adverse reaction when interacting with living cells or the body. This includes being non-toxic and non-inflammatory. Accordingly, a bioink may be modified or functionalised according to the cell type it is intended for use with. This is due to the unique needs of different cell types, such as the necessary nutrients, growth factors, and physical characteristics that enable the cells to thrive and function. For example, the bioink may be modified with thiol moieties. In some embodiments, the bioink is selected from gelatin methacryloyl (GelMA), gelatin, collagen, silk fibroin, alginate, nanofibrillated cellulose, hyaluronic acid, poly(caprolactone-co-ethyl ethylene phosphate), polyethylene glycol diacrylate (PEGDA) or a combination thereof. In some embodiments, the bioink is GelMA. In some embodiments, the bioink is capable of being photo-chemically crosslinked.

In some embodiments, the bioink is a combination of GelMa and PEGDA.

In some embodiments, the hydrogel ink comprises:
a) lipid particles;
b) polynucleotides loaded within the lipid particles; and
c) gelatin methacryloyl (GelMA).

In some embodiments, the bioink is about 10% wt/wt to about 40% wt/wt relative to the hydrogel ink. In other embodiments, the concentration is about 10% wt/wt to about 38% wt/wt, about 10% wt/wt to about 36% wt/wt, about 10% wt/wt to about 34% wt/wt, about 10% wt/wt to about 32% wt/wt, about 10% wt/wt to about 30% wt/wt, about 10% wt/wt to about 28% wt/wt, about 10% wt/wt to about 26% wt/wt, about 10% wt/wt to about 24% wt/wt, about 10% wt/wt to about 22% wt/wt, about 10% wt/wt to about 20% wt/wt, about 12% wt/wt to about 20% wt/wt, about 14% wt/wt to about 20% wt/wt, or about 15% wt/wt to about 20% wt/wt. In other embodiments, the concentration is about 15% wt/wt, or about 20% wt/wt.

In some embodiments, the bioink comprises PEGDA at a concentration of about 0.1% wt/wt to about 1% wt/wt. In other embodiments, the concentration is about 0.2% wt/wt to about 1% wt/wt, about 0.3% wt/wt to about 1% wt/wt, about 0.4% wt/wt to about 1% wt/wt, about 0.5% wt/wt to about 1% wt/wt, about 0.6% wt/wt to about 1% wt/wt, about 0.7% wt/wt to about 1% wt/wt, or about 0.8% wt/wt to about 1% wt/wt. It was found that PEGDA helps to stabilise the scaffold structure, and a PEGDA amount that is higher than 1% is detrimental to spinal cord tissues, causing serious degeneration of tissues in vivo.

The hydrogel ink may comprise other components. For example, the hydrogel ink may comprise an aqueous medium. The aqueous medium may be added to modulate the physical properties of the hydrogel ink. For example, the viscosity and hence flowability of the hydrogel ink may be modulated. The aqueous medium may also slow down the crosslinking reaction of the hydrogel ink, thus allowing it to be 3D printable.

The term ‘aqueous medium’ used herein refers to a water based solvent or solvent system, and which comprises of mainly water. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also falls within this definition.

In some embodiments, the aqueous medium is phosphate-buffered saline. In some embodiments, the aqueous medium is about 1% wt/wt to about 10% wt/wt relative to the hydrogel ink. In other embodiments, the concentration is about 1% wt/wt to about 9% wt/wt, about 1% wt/wt to about 8% wt/wt, about 1% wt/wt to about 7% wt/wt, about 1% wt/wt to about 6% wt/wt, about 1% wt/wt to about 5% wt/wt, about 1% wt/wt to about 4% wt/wt, or about 1% wt/wt to about 3% wt/wt. In other embodiments, the concentration is about 2% wt/wt.

In some embodiments, the hydrogel ink further comprises a photoinitiator. Photoinitiators are molecules that absorb photons upon irradiation with light and form reactive species out of the excited state, which initiate consecutive reactions. The initiating species may be radicals, cations, or anions. Radical photoinitiators are commercially available in large number from companies such as Ciba Specialties (trade names Irgacure and Darocure), Lamberti (Esacure), BASF (Lucirin), and many others. Cationic photoinitiators are mainly sulfonium salts, from Union Carbide (Cyracure), Degussa (Degacure) as well as iodonium salts from General Electric and iron complexes from Ciba. The photoinitiator may be lithium phenyl (2,4,6-trimethylbenzoyl) phosphinate (LAP). LAP is a free radical photo-initiator used to initiate free radical chain polymerization upon light exposure, and may be combined with GelMA to produce a photopolymer for use in bioprinting. Other commercially available photoinitiators include dimethoxyphenylacetophenone, α-hydroacetophenone, α-aminoacetophenone, titanocene (CG-784), benzoylphosphineoxide, and bisbenzoylphosphineoxide.

In some embodiments, the photoinitiator is about 0.1% wt/wt to about 1% wt/wt relative to the hydrogel ink. In other embodiments, the concentration is about 0.1% wt/wt to about 0.9% wt/wt, about 0.1% wt/wt to about 0.8% wt/wt, about 0.1 % wt/wt to about 0.7% wt/wt, about 0.1% wt/wt to about 0.6% wt/wt, about 0.1 % wt/wt to about 0.5% wt/wt, about 0.1% wt/wt to about 0.4% wt/wt, about 0.1 % wt/wt to about 0.3% wt/wt, or about 0.1% wt/wt to about 0.2% wt/wt.

In some embodiments, the hydrogel ink further comprising a photoquencher (or scavenger). The photoquencher quenches the photo-absorbance capacity of impurities present in the lipid particles. For example, haemoglobin may be present, which has a broad absorbance spectrum, ranging from 340 to 440 nm and from 540 to 580 nm, with a prominent peak at 420 nm. Due to this absorption, the photoinitiator may not be able to efficiently absorb the photon, and this may slow down the crosslinking reaction and thus the polymerisation of the hydrogel. The photoquencher may be curcumin. In this regard, curcumin interacts as an iron chelator with hem group of haemoglobin, allowing an energy transfer between n-electron rich curcumin and haemoglobin as an electron deficient compound.

In some embodiments, the photoquencher is about 0.1% wt/v to about 10% wt/v relative to the hydrogel ink. In other embodiments, the concentration is about 0.1% wt/v to about 9% wt/v, about 0.1% wt/v to about 8% wt/v, about 0.1% wt/v to about 7% wt/v, about 0.1% wt/v to about 6% wt/v, about 0.1% wt/v to about 5% wt/v, about 0.1% wt/v to about 4% wt/v, about 0.1% wt/v to about 3% wt/v, about 0.1% wt/v to about 2% wt/v, or about 0.1% wt/v to about 1% wt/v. In some embodiments, the photoquencher is curcumin, at about 0.7% wt/v to about 7.5% wt/v.

In some embodiments, the photoquencher is about 0.1% wt/wt to about 10% wt/wt relative to the hydrogel ink. In other embodiments, the concentration is about 0.1% wt/wt to about 9% wt/wt, about 0.1% wt/wt to about 8% wt/wt, about 0.1% wt/wt to about 7% wt/wt, about 0.1% wt/wt to about 6% wt/wt, about 0.1% wt/wt to about 5% wt/wt, about 0.1% wt/wt to about 4% wt/wt, about 0.1% wt/wt to about 3% wt/wt, or about 0.1% wt/wt to about 2% wt/wt, or about 0.1% wt/wt to about 1% wt/wt. In some embodiments, the photoquencher is curcumin, at about 0.7% wt/wt to about 7.5% wt/wt.

In some embodiments, the photoquencher is about 1×10⁻⁴% wt/wt to about 1×10⁻²% wt/wt relative to the hydrogel ink. In other embodiments, the concentration is about 1×10⁻⁴% wt/wt to about 9×10⁻³% wt/wt, about 1×10⁻⁴% wt/wt to about 8×10⁻³% wt/wt, about 1×10⁻⁴% wt/wt to about 7×10⁻³% wt/wt, about 1×10⁻⁴% wt/wt to about 6×10⁻³% wt/wt, about 1×10⁻⁴% wt/wt to about 5×10⁻³% wt/wt, about 1×10⁻⁴% wt/wt to about 4×10⁻³% wt/wt, about 1×10⁻⁴% wt/wt to about 3×10⁻³% wt/wt, about 1×10⁻⁴% wt/wt to about 2×10⁻³% wt/wt, about 1×10⁻⁴% wt/wt to about 1×10⁻³% wt/wt, about 1×10⁻⁴% wt/wt to about 9×10⁻⁴% wt/wt, about 1×10⁻⁴% wt/wt to about 8×10⁻⁴% wt/wt, about 1×10⁻⁴% wt/wt to about 7×10⁻⁴% wt/wt, about 1×10⁻⁴% wt/wt to about 6×10⁻⁴% wt/wt, about 1×10⁻⁴% wt/wt to about 5×10⁻⁴% wt/wt, about 1×10⁻⁴% wt/wt to about 4×10⁻⁴% wt/wt, or about 1×10⁻⁴% wt/wt to about 3×10⁻⁴% wt/wt. In some embodiments, the photoquencher is curcumin, at about 7×10⁻⁴% wt/wt to about 7×10⁻⁴% wt/wt.

In some embodiments, the photoquencher is about 1×10⁻⁴% wt/v to about 1×10⁻²% wt/v relative to the hydrogel ink. In other embodiments, the concentration is about 1×10⁴% wt/v to about 9×10⁻³% wt/v, about 1×10⁻⁴% wt/v to about 8×10⁻³% wt/v, about 1×10⁻⁴% wt/v to about 7×10⁻³% wt/v, about 1×10⁻⁴% wt/v to about 6×10⁻³% wt/v, about 1×104% wt/v to about 5×10⁻³% wt/v, about 1×10⁻⁴% wt/v to about 4×10⁻³% wt/v, about 1×10⁻⁴% wt/v to about 3×10⁻³% wt/v, about 1×10⁻⁴% wt/v to about 2×10⁻³% wt/v, about 1×10⁻⁴% wt/v to about 1×10⁻³% wt/v, about 1×10⁻⁴% wt/v to about 9×10⁻⁴% wt/v, about 1×10⁻⁴% wt/v to about 8×10⁻⁴% wt/v, about 1×10⁻⁴% wt/v to about 7×10⁻⁴% wt/v, about 1×10⁻⁴% wt/v to about 6×10⁻⁴% wt/v, about 1×10⁻⁴% wt/v to about 5×10⁻⁴% wt/v, about 1×10⁻⁴% wt/v to about 4×10⁻⁴% wt/v, or about 1×10⁻⁴% wt/v to about 3×10⁻⁴% wt/v. In some embodiments, the photoquencher is curcumin, at about 7×10⁻⁴% wt/v to about 7×10⁻⁴% wt/v.

In some embodiments, the hydrogel ink further comprises a drug. The drug may be a protein, or a polynucleotide. For example, the drug may be a miRNA, siRNA, shRNA, antisense oligonucleotide, antibody, peptide, protein or a chemotherapy drug. The drug may be encapsulated within the lipid particle, and/or within the matrix of the hydrogel ink. The peptide or protein may be a growth factor or chemoattractant.

In some embodiments, the hydrogel ink is 3D printable. Examples of 3D printing approaches include, but are not limited to, digital light processing 3D printing, stereolithogrphy 3D printing, and extrusion printing.

The present disclosure also provides a hydrogel, comprising:
a) lipid particles;
b) polynucleotides loaded within the lipid particles; and
c) a bioink matrix;
   wherein the lipid particles are homogenously dispersed within the bioink matrix.

The homogeneity of the lipid particles may be controlled by altering the speed of crosslinking of the bioink.

Various printing techniques may be used to form the hydrogel's structure. For example, the hydrogel ink may be used in layer-by-layer printing to provide a hydrogel. This method enhance scaffold attachment of cells and also provide structural integrity to the hydrogel. In addition, when 3D printing, an over-crosslinked sacrificial layer may be printed as a base to allow for a firm attachment of the scaffold to the printer holder. Alternatively, exposure time or light intensity may be varied or reduced in a controlled, layer-by-layer manner to minimize over-crosslinking in the preceding layers. The scaffold may also be 3D printed with holding pillars between the scaffold layers and the sacrificial layer, which may be subsequently removed after printing.

### In Some Embodiments, the Hydrogel Comprises:

a) lipid particles;
b) polynucleotides loaded within the lipid particles; and
c) a gelatin methacryloyl matrix;
   wherein the lipid particles are homogenously dispersed within the gelatin methacryloyl matrix.

In some embodiments, the lipid particles are about 2% wt/wt to about 10% wt/wt relative to the hydrogel. In other embodiments, the concentration is about 4% wt/wt to about 10% wt/wt, about 6% wt/wt to about 10% wt/wt, about 8% wt/wt to about 10% wt/wt, about 2% wt/wt to about 8% wt/wt, about 2% wt/wt to about 6% wt/wt, about 2% wt/wt to about 5% wt/wt, or about 2% wt/wt to about 4% wt/wt. In other embodiments, the concentration is about 4% wt/wt.

In some embodiments, the bioink is about 10% wt/wt to about 40% wt/wt relative to the hydrogel. In other embodiments, the concentration is about 10% wt/wt to about 38% wt/wt, about 10% wt/wt to about 36% wt/wt, about 10% wt/wt to about 34% wt/wt, about 10% wt/wt to about 32% wt/wt, about 10% wt/wt to about 30% wt/wt, about 10% wt/wt to about 28% wt/wt, about 10% wt/wt to about 26% wt/wt, about 10% wt/wt to about 24% wt/wt, about 10% wt/wt to about 22% wt/wt, about 10% wt/wt to about 20% wt/wt, about 12% wt/wt to about 20% wt/wt, about 14% wt/wt to about 20% wt/wt, or about 15% wt/wt to about 20% wt/wt. In other embodiments, the concentration is about 15% wt/wt, or about 20% wt/wt.

In some embodiments, the photoinitiator is about 0.1% wt/wt to about 1% wt/wt relative to the hydrogel. In other embodiments, the concentration is about 0.1% wt/wt to about 0.9% wt/wt, about 0.1% wt/wt to about 0.8% wt/wt, about 0.1% wt/wt to about 0.7% wt/wt, about 0.1% wt/wt to about 0.6% wt/wt, about 0.1% wt/wt to about 0.5% wt/wt, about 0.1% wt/wt to about 0.4% wt/wt, about 0.1% wt/wt to about 0.3% wt/wt, or about 0.1% wt/wt to about 0.2% wt/wt.

In some embodiments, the hydrogel is 3D printed. In some embodiments, the hydrogel is formed as a multichannel scaffold.

The present disclosure also provides a method of 3D printing a hydrogel as disclosed herein, comprising printing the hydrogel ink and crosslinking the hydrogel ink in order to form the hydrogel. The crosslinking may be performed under 405 nm light. The crosslinking may be performed for at least 60 sec, or at least 80 sec. The crosslinking may be performed at the same time as the printing step.

Due to the absorption peaks of haemoglobin in the RBCEVs, the exposure time required to form a stable scaffold may be higher than other EVs.

The present disclosure also provides a method of transfecting cells, comprising the step of contacting the hydrogel as disclosed herein with the cells. When the hydrogel is formed as a multichannel scaffold, the cells may be positioned within the channels. For example, a cell may be position within each channel.

In some embodiments, the method is characterised by a EV loading to 1×10⁵ cells of about 10 μg to about 100 μg. In other embodiments, the EV loading is about 10 μg to about 90 μg, about 10 μg to about 80 μg, about 10 μg to about 70 μg, about 10 μg to about 60 μg, about 10 μg to about 50 μg, about 10 μg to about 40 μg, about 10 μg to about 30 μg, or about 10 μg to about 20 μg.

In some embodiments, the method is characterised by a polynucleotide loading to 1×10⁵ cells of about 150 ng to about 400 ng. In other embodiments, the polynucleotide loading is about 150 ng to about 380 ng, about 150 ng to about 360 ng, about 150 ng to about 340 ng, about 150 ng to about 320 ng, about 150 ng to about 300 ng, about 150 ng to about 280 ng, about 150 ng to about 260 ng, about 160 ng to about 260 ng, about 180 ng to about 260 ng, or about 200 ng to about 260 ng. In other embodiments, the polynucleotide loading is about 250 ng.

In some embodiments, the method is characterised by a transfection rate of at least about 50%. In other embodiments, the transfection rate is at least about 55%, about 60%, about 65%, about 70%, about 75%, about 80%, about 85%, or about 90%.

The present disclosure also provides a method of treating a neural condition or disease in a subject in need thereof, comprising administering a therapeutically effective amount of the hydrogel as disclosed herein to a subject.

The present disclosure also provides a hydrogel as disclosed herein for use in treating a neural condition or disease.

The present disclosure also provides a use of the hydrogel as disclosed herein in the manufacture of a medicament for the treatment a neural condition or disease.

The hydrogel may be used to treat a disease or condition associated with the central nervous system. For example, pathological conditions related to the central nervous system and/or the peripheral nervous system may be treated. In some embodiments, the neural condition or disease is selected from spinal cord injury, brain injury, ischemic stroke, and sclerosis. In some embodiments, the neural condition or disease is selected from spinal cord injury, subacute ischemic stroke, traumatic brain injury and injury to the peripheral nervous system. In some embodiments, the neural condition or disease is an injury within the nervous system characterised by a space, the space for implanting a scaffold. The hydrogel may be capable for promoting neuroregenerative processes relevant to SCI therapy, including neurogenesis, adaptive neural plasticity, and remyelination and regeneration of injured axons over long-distances, glial scarring and cortical lesion volume.

The present disclosure also provides a method of regenerating a soft tissue in a subject in need thereof, comprising administering a therapeutically effective amount of the hydrogel as disclosed herein to a subject.

The present disclosure also provides a hydrogel as disclosed herein for use in regenerating a soft tissue.

The present disclosure also provides a use of the hydrogel as disclosed herein in the manufacture of a medicament for the regeneration of a soft tissue.

Soft tissues connect, support, and surround the different body organs. They can be found in most parts of the body. Soft tissues include fat, muscle, nerves, myelin, blood vessels, ligaments, tendons, and other fibrous tissues. For example, for nerve tissue regeneration, all component of the spinal cord tissue may be regenerated, including astrocytes, microglia, neurons, and oligodendrocytes. The hydrogel scaffold may be used to treat other soft tissue by changing the drug targets. For example, for cardiovascular diseases, proteins specific to cardiovascular applications (e.g. VEGF, PDGF), nucleic acids specific to signaling pathways critical to cardiovascular applications may be used. The hydrogel may also be used for skin regeneration.

In some embodiments, the nerve is regenerated.

### EXAMPLES

In general, nucleic acid and optionally drugs were loaded into EVs that were extracted from cells. Thereafter, the nucleic acid loaded-EVs were mixed with bioink (e.g. GelMA) solution for 3D printing (e.g. digital light processing). The efficacy of these scaffolds that encapsulated nucleic acid loaded-EVs were then tested in vitro and in vivo. As a proof of concept, we have chosen the more difficult models, i.e. RNA incorporation (since RNA is less stable than DNA); and the central nervous system (since primary neural cells are more difficult to maintain cell viability and be transfected by nucleic acids than other cell types/cell lines).

It is thus demonstrated that:
1) nucleic acids loaded-EVs may be loaded or encapsulated into tissue engineered scaffolds, while retaining structure and bioactivity of nucleic-acids loaded EVS.
2) sustained and controlled delivery of nucleic acids loaded EVs using tissue engineered scaffolds is achievable.
3) the nucleic acids-loaded EVs may be exposed to additive manufacturing methods like 3D printing, which involves the exposure of nucleic acids-loaded EVs to light exposure, photochemical crosslinking and scaffold fabrication at ambient conditions, without significant degradation of the nucleic acids-loaded EVs.
4) difficult to transfect primary mammalian cells may be transfected with nucleic acids loaded EVs by scaffold-mediated delivery approach.

### Method

Transfection efficiency of RNA loaded-EVs was first tested in different types of central nervous system glia cells and neurons. Thereafter, small non-coding RNAs (siRNA, microRNA cocktail) were loaded into EVs, which was then mixed into GelMA solution for photo crosslinking-based 3D printing. Before transplantation into the rodent spinal cord injury model, the printed scaffolds were also tested in vitro using primary rat oligodendrocytes progenitor cells (OPCs) to demonstrate the retention of bioactivity of RNA-EVs, as well as investigate the function of RNA-EVs on OPCs differentiation and myelination.

### EVs Extraction and miRNA Loading

EVs were extracted from the blood samples of healthy human donors with informed consent were obtained from Innovative Research. EVs (50 μg) were transfected with 1 μg of miRNA cocktail using EVfect transfection reagent (Jotbody) according to the manufacturer's protocols.

In general, the method of EV extraction and nucleic acid loading comprises purifying extracellular vesicles (EVs) from red blood cells (RBCs) using ultracentrifugation with a sucrose cushion, wherein the RBCs have been treated overnight with calcium ionophore, followed by RNA loading into the EVs using specialized reagents for loading nucleic acids into EVs, resulting in RNA-loaded EVs. EVs can also be alternatively loaded with RNA via electroporation. This is disclosed, for example, in US Ser. No. 15/678,363, the disclosure of which is herein incorporated by reference.

### EVs/GelMA Scaffold Fabrication

For 250 μL bioink, 187.5 μL 20% GelMA, 16.67 μL 3% LAP, 5.83 μL PBS, and 40 L EVs (20 mg/mL) were mixed together and incubated at 37 °C before printing. Under 405 nm light for 80 s, the scaffolds crosslinked well with clear internal microchannel structures. Theoretically, one scaffold finally should consist of 15% GelMA, 0.2% LAP, 9.6 μg EVs (264 ng miRNA).

### Results

### Primary Mammalian Astrocytes and OPCs Could Efficiently Take up EVs

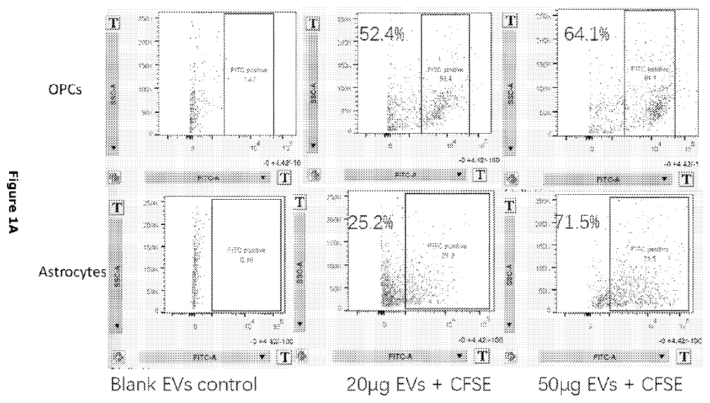

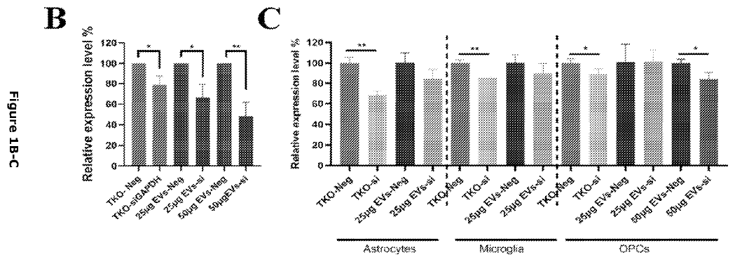

Both primary rat oligodendrocyte progenitor cells (OPCs) and astrocytes could uptake fluorescent nucleic acids loaded-EVs efficiently in a dosage-dependent trend. At a 50 μg RNA-EVs/1×10⁵ cells ratio, ≥64.1% OPCs and ≥71.5% astrocytes successfully took up RNA-EVs, as shown in FIG. 1A.

### Sigapdh-EVs Could Effectively Knockdown GAPDH Expression in Neurons and Glial Cells

As shown in FIG. 1B and C, GAPDH gene expression in all types of neural cells could be downregulated by siGAPDH-EVs treatment at 25 μg/1×10⁵ cells ratio, which contained 500 ng of GAPDH siRNA. The ability to downregulate the expression of GAPDH, a housekeeping gene, is significant because this is an endogenous gene that exists in high abundance in the cell. Being able to knockdown such a gene would suggest high possibility in modulating all other targets that exist in lower abundance. When compared with commercial transfection reagent, TKO, similar knockdown efficiencies were observed. Specifically, in neurons, there was a dosage-dependent significant knockdown effect of siGAPDH-EVs. There was significant knockdown trend of 25 μg of EVs in neurons, astrocytes, and microglia. However, 50 μg RBCEVs-siRNA was required to induce significant knockdown effect on OPCs. Therefore, dosage of drugs required is cell-type dependent.

### Sigapdh-EVs/GelMA Scaffold Could Effectively Knockdown Gapdh Expression in OPCs

To investigate if the siGAPDH-EVs could be released and still be functional after being incorporated into scaffolds, the nucleic acid loaded-EVs incorporated scaffolds were placed in a transwell insert and incubated with OPCs for 5 days to evaluate the gene knockdown effect. FIG. 1D shows the process of experiment design. FIG. 1E represents the GAPDH expression level after co-culture, which showed that siGAPDH-EVs/GelMA scaffold could effectively knockdown GAPDH expression in OPCs at a ratio of ~250 ng siRNA/1×10⁵ cells. Moreover, the knockdown effect is even more robust than TKO-siGAPDH complex.

miR-219/miR-338-EVs/GelMA Scaffold Effectively Supported Oligodendroglial Lineage Cells

Importantly, miRNA-EVs/GelMA scaffold could successfully support oligodendroglial lineage cells, as indicated by FIG. 1F. At day 3 of culture, Olig2 average intensity in the OPCs co-cultured with miRNA-EVs/GelMA scaffold showed significance when compared to negative RNA control group. Besides, Olig2 positive cells at day 3 of OPCs culture reached ~40%, which is significantly higher than negative RNA control (~15%).

### In Vivo Studies

The in vivo functionality of our RBCEV-incorporated scaffolds, using the rat complete transection spinal cord injury model, was explored. We focused on two key aspects: 1) the extent of cellular uptake of RBCEVs by local tissue cells, using 3D printed scaffolds that incorporated fluorescently labeled RBCEVS (Cy5-CFSE EVs), and 2) the biofunctionality of the scaffolds by analyzing the extent of oligodendrocytes maturation in response to 3D printed scaffolds that incorporated RBCEV-miR-219/miR-338) complexes, where miR-219/miR-338 has been reported by us and others to enhance oligodendrocyte differentiation and maturation.

The RBCEVs were complexed with Cy5-labeled oligodeoxynucleotide (Cy5-ODN) and subsequently incorporated into 3D printed scaffolds. Briefly, 1 mg of EVs was mixed into a 10% w/v methacrylated gelatin (GelMA) mixture with 0.1% w/w LAP. The Digital Light Processing (DLP) 3D printing technique was utilized, deploying 300 μL of ink per print with the following parameters: 25 mW/cm² intensity, layer-by-layer deposition with a 500 μm layer thickness, and a 10 s exposure per layer for photo-crosslinking. Post-printing, the scaffolds were incubated at 37 °C with 100 rpm shaking overnight to eliminate residual ink within the channels. The scaffolds were then transplanted into a rat spinal cord injury model (2 mm complete transection at T9-10 level). Seven days post-transplantation, the rats were euthanized, and the affected spinal cord regions were fixed in 4% paraformaldehyde (PFA) and prepared for immunostaining analysis.

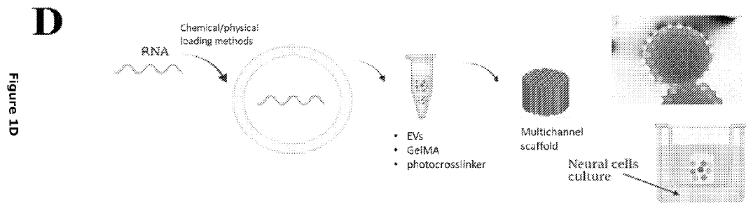

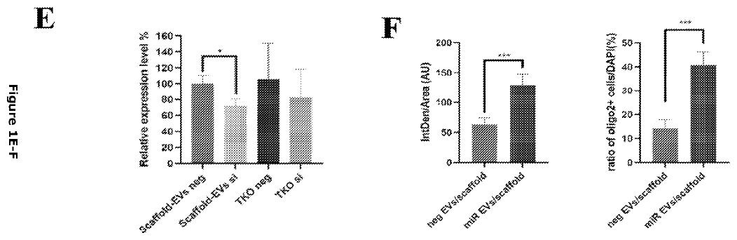

The transplanted scaffolds (composed of 10% GelMA with 400 μm channels and 100 μm wall thickness) demonstrated localized delivery of EVs up to 3 mm from the scaffold-tissue interface after 7 days (FIG. 2). The scaffolds continued to function as reservoirs for sustained delivery of EVs to the surrounding spinal cord tissues. Notably, the EVs within the scaffold showed diminished Cy5 signals. A time delay was observed in the peak positions of CFSE and Cy5, indicating the process of EV internalization and release-the Cy5 peak lagged behind the CFSE peak by 1.5-2mm. Confocal microscopy revealed that mature oligodendrocytes (CC1-positive cells) displayed significant internalization of EVs and Cy5 signals, both within the scaffold channel area and in the furthest areas of EV distribution.

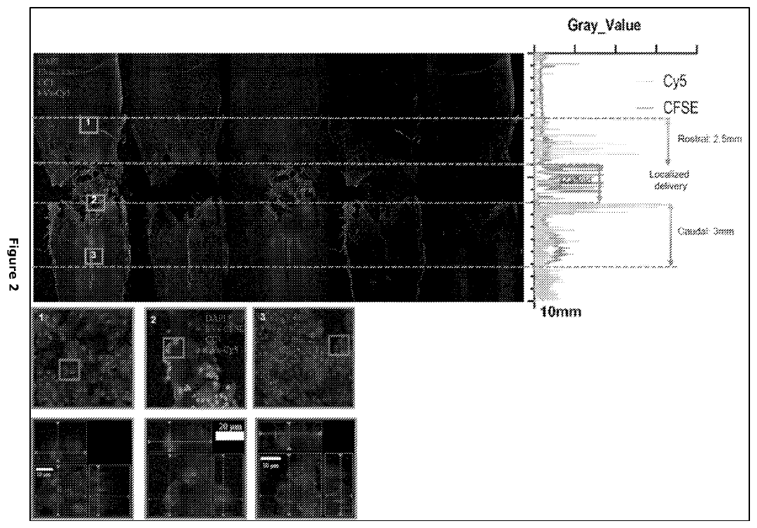

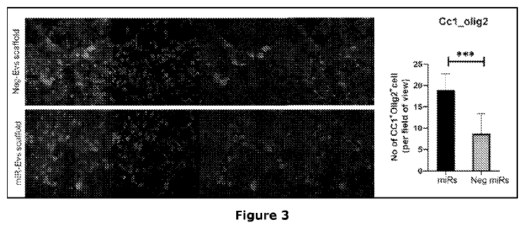

The RBCEV-miR-219/miR-338 incorporated scaffolds significantly increased the number of CC1-positive mature oligodendrocytes as compared to the negative control, which comprised of RBCEV-scrambled miR incorporated scaffolds (FIG. 3). The results suggest the biofunctionality of the RBCEV-miR-219/miR-338 that were released from the scaffolds in promoting oligodendrocyte maturation in vivo.

Our study demonstrates that the RBCEV-miR- 219/miR-338 3D printed scaffold can achieve sustained and localized delivery of biofunctional RBCEV-miRs up to 2.5-3 mm from the implant site and into the surrounding spinal cord tissue within 7 days of transplantation. Moreover, the RBCEV-miR-219/miR-338 incorporated scaffolds exhibited a potent maturation-promoting effect on oligodendrocytes in vivo, highlighting its therapeutic potential for spinal cord injury treatment.

### Discussion and Conclusion

RBCEVs is a promising RNA vector for nucleic acid delivery into neuronal cells, which is still functional after incorporated into GelMA scaffolds to induce downstream cellular response. RNA-RBCEVs/GelMA scaffold showed that they may be used in gene delivery for SCI treatment, and also be used in nerve regeneration and remyelination.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A hydrogel ink, comprising:
a) lipid particles;
b) polynucleotides loaded within the lipid particles; and
c) a bioink.

2. The hydrogel ink according to claim 1, wherein the lipid particles are extracellular vesicles.

3. The hydrogel ink according to claim 1 , wherein the lipid particles are extracellular vesicles derived from red blood cells (erythrocytes), immune cells, endothelial cells, liver cells, epithelial cells, stem cells, Schwann cell, or a combination thereof.

4. The hydrogel ink according to claim 1, wherein the lipid particles are characterised by an average particle size of about 100 nm to about 400 nm.

5. The hydrogel ink according to claim 1, wherein the lipid particles are about 0.01% wt/wt to about 10% wt/wt relative to the hydrogel ink.

6. The hydrogel ink according to claim 1, wherein the polynucleotides are selected from deoxyribonucleic acid (DNA), small non-coding RNA, or small interfering RNA (siRNA);
wherein the polynucleotides are about 0.5% wt/wt to about 10% wt/wt relative to the lipid particles.

7. (canceled)

8. The hydrogel ink according to claim 1, wherein the bioink is selected from gelatin methacryloyl (GelMA), gelatin, collagen, silk fibroin, alginate, nanofibrillated cellulose, hyaluronic acid, poly(caprolactone-co-ethyl ethylene phosphate), polyethylene glycol diacrylate (PEGDA) or a combination thereof;
wherein the bioink is about 10% wt/wt to about 40% wt/wt relative to the hydrogel ink.

9. (canceled) .

10. The hydrogel ink according to claim 1, wherein the hydrogel ink further comprising a photoinitiator, wherein the photoinitiator is about 0.1% wt/wt to about 1% wt/wt relative to the hydrogel ink; and/or
a photoquencher, wherein the photoquencher is about 1×10⁻⁴% wt/wt to about 1×10⁻²% wt/wt relative to the hydrogel ink.

11. (canceled)

12. The hydrogel ink according to claim 1, wherein the hydrogel ink further comprises a drug.

13. The hydrogel ink according to claim 1, wherein the hydrogel ink is 3D printable.

14. A hydrogel, comprising:
a) lipid particles;
b) polynucleotides loaded within the lipid particles; and
c) a bioink matrix;
wherein the lipid particles are homogenously dispersed within the bioink matrix.

15. The hydrogel according to claim 14, wherein the hydrogel is formed as a multichannel scaffold.

16. A method of transfecting cells, comprising the step of contacting the hydrogel according to claim 14 with the cells.

17. The method according to claim 16, wherein the method is characterised by a EV loading to 1×10⁵ cells of about 10 μg to about 100 μg.

18. The method according to claim 16, wherein the method is characterised by a polynucleotide loading to 1×10⁵ cells of about 150 ng to about 400 ng.

19. The method according to claim 16, wherein the method is characterised by a transfection rate of at least about 50%.

20. A method of treating a neural condition or disease in a subject in need thereof, comprising administering a therapeutically effective amount of the hydrogel according to claim 14 to a subject.

21. (canceled)

22. (canceled)

23. The method, according to claim 20, wherein the neural condition or disease is selected from spinal cord injury, brain injury, ischemic stroke, and sclerosis.

24. A method of regenerating a soft tissue in a subject in need thereof, comprising administering a therapeutically effective amount of the hydrogel according to claim 14 to a subject.

25. (canceled)

26. (canceled)

27. The method, according to claim 24, wherein the soft tissue is a nerve, cardiovascular tissue or skin.
